(19) United States
(12) Patent Application Publication
(10) Pub. No.: US 2022/0277820 A1
(43) Pub. Date: Sep. 1, 2022

(54) PHARMACY MATCHING METHOD AND PHARMACY MATCHING SYSTEM USING THE SAME

(71) Applicant: PALM FIRM CORPORATION, Seoul (KR)

(72) Inventors: Minsung KWON, Seoul (KR); Hochun CHOI, Seoul (KR)

(73) Assignee: PALM FIRM CORPORATION, Seoul (KR)

(21) Appl. No.: 17/624,665

(22) PCT Filed: Jun. 29, 2020
(86) PCT No.: PCT/KR2020/008451

§ 371 Date: Jan. 4, 2022

(30) Foreign Application Priority Data
Jul. 5, 2019 (KR) 10-2019-0081438

(51) Int. Cl. G16H 20/10

(52) U.S. Cl. CPC G16H 20/10

(57) ABSTRACT

A pharmacy-matching method includes a transmitting/receiving unit of service server receives a pharmacy search request information transmitted from a user instrument; a pharmacy information management department of the service server extracts at least one pharmacy information included in the pharmacy search request information; a user management department of the service server transmits at least one pharmacy information to the user instrument; the user management department requests prescribing drug information from the user instrument and receives the requested information; the matching pharmacy management department transmits the information included in the prescribing drug information to pharmacy terminal included in at least one pharmacy information, transmits information required for confirmation, and receives acceptance information about whether it can be prepared; and the matching pharmacy management department confirms the requested information to transmit at least one pharmacy information permitting the preparation to the user instrument.

### TECHNICAL FIELD

The present invention relates to a pharmacy-matching method, and more particularly, to provide a pharmacy-matching method with high user accessibility in relation to prescription medicine.

### BACKGROUND ART

The separation of drug prescribing and dispensing system was introduced to rationally distribute diagnosis and pharmacology of the disease to physicians and pharmacists who are experts in their respective fields. Specifically, when a physician attempts to use a medication to a patient by means of a treatment, the physician advises the patient the prescription, and the pharmacy means an instruction to prepare and manage the medication according to the prescription. That is, after a physician who is a medical professional diagnoses the symptoms of a patient and most suitably prescribes a pharmaceutical to be used for the treatment of the patient, the pharmacy is introduced to provide a high-quality medical service by preparing, encouraging and treating the pharmaceutical according to the prescription.

Separation of drug prescribing and dispensing was performed on Aug. 1, 2000 with a life span of one month under the effect of reducing the misuse of drugs up to 5-7 times the developed world and providing accurate medical service. The separation of drug prescribing and dispensing system are excluded from agricultural and detoxified areas that have no pharmacology, with the exception of emergency patients, hospitalized patients, and patients that can be damaged by themselves or others.

Although the separation of drug prescribing and dispensing system reduces the misuse of drugs and provides accurate medical service, consumers who have received the prescription are not only directly receiving the prescription issued by the medical agency and then being submitted to the pharmacy to prepare the separation of drug prescribing and dispensing, but also because the prescription information is difficult to be transmitted directly between hospitals and pharmaceutics, there is a problem that in the case of a pharmacy country adjacent to a large hospital such as a general hospital, it takes a lot of time for the prescription to be filed and to receive the prepared separation of drug prescribing and dispensing, and patients who are uncomfortable to the body have to wait for a long time in the medical country. In the case of a consumer who visits to a hospital for receiving treatment, the consumer's discomfort has been greatly increased as compared to the time at which a diagnosis and preparation has been made in a previous hospital because the hospital's entry has not received a one-stop service at a place where medical treatment and prescription are made. In particular, in the case where an atmosphere is intended to receive a drug in a living area of a patient, an area where the patient is located, or the like, rather than in an unattractive pharmacy around many hospitals, a drug on the prescription or a drug replacing it is not provided in an hometown pharmacy, and thus the drug cannot be purchased in some cases.

In the case of small-scale pharmacy, chemicals that are generally not used in large quantities will build up unnecessary inventory (unused inventory) and consequently lead to space utilization issues and financial burdens due to inventory. On the other hand, it is not easy to find the pharmacy itself in the hospital for a consumer who is not frequently used for a pharmacy, or who is intended to use a pharmacy in an area of a low frequency. Even if a pharmacy is being sought, it is difficult to identify in advance whether or not a general consumer who is deficient in knowledge and information about a drug has the drug required for prescription in that pharmacy. Due to such a practical problem and the like, it appears that discomfort accompanying the separation of drug prescribing and dispensing is further weighted.

Accordingly, if the user can check in advance whether the prescribed drug can be provided at the desired pharmacy, the user's convenience will be greatly increased. Even in the case of a pharmacy, if the user actually takes a period of time until the drug is received, the drug may be ready for that time and the user's convenience may be increased. As a result, if it is possible to rationally adjust the cost of the user's preparation and the time of preparing the drug in the pharmacy, only the drug required for the preparation can be used in the preparation by being wet, which will increase the efficiency in the operating pharmacy since the space utilization and inventory burden of the drug can be reduced.

### PRIOR ART LITERATURES

### Patent Literature

(Patent Document 1) Patent Publication No. 10-2016-0038661 (2016.04.07.)

### DETAILED DESCRIPTION

### Technical Problem

The pharmacy-matching method according to an embodiment of the present invention aims to allow a user to prepare a desired prescribing drug at a desired time in the pharmacy at a position where the user's convenience can be maximized.

The objects of the present invention are not limited to those mentioned above, and further objects which are not mentioned will be apparent to those skilled in the art from the following description.

### Means to Solve the Problem

A pharmacy-matching method according to one aspect of the present invention for solving the above technical problem includes: receiving, by the data transmitting/receiving unit of the service server, the pharmacy search request information transmitted from the user instrument; extracting, by a pharmacy information management department of the service server, at least one pharmacy information included in the pharmacy search request information; transmitting, by a user management department of the service server, at least one pharmacy information to the user instrument; receiving, by the user management department, requesting prescribing information from a user instrument and receiving the information; The matching pharmacy management department of the service server transmits the prescribing drug information included in the prescribing information to the pharmacy terminal included in the at least one pharmacy information to transmit information required for confirmation to check whether dispensing is possible and receive information on acceptance of dispensing availability to do; and the matching pharmacy management department confirms the requested information to transmit at least one pharmacy information permitting the preparation to the user instrument.

The prescribing information is prescription image information or prescription text information, the step of requesting and receiving said prescribing information further comprises: if the prescribing information is the prescription image information, the service server transmits the prescription image information to the optical character recognition system server, requests to extract prescribing drug information to extract the prescribing drug information included in the prescription image information from the optical character recognition system, and receives the extracted prescribing drug information.

Here, the pharmacy search request information includes the current position information, destination information, and information on the movement means transmitted from the user instrument, and the step of extracting the pharmacy information is a user generated using the current position information and the destination information. The method may further include extracting pharmacy information within a predetermined radius of the movement path of the device.

Here, the movement means included in the information on the movement means may be a automobile, public traffic, bicycle, or walking.

Here, the step of extracting the pharmacy information within a predetermined radius range is provided to extract the pharmacy information using the point of departure, destination or transfer information of the user instrument when the movement means is public traffic, and when the movement means is a automobile, a automobile It may be provided to extract pharmacy information using the driving path of, but to select and extract pharmacy information located on the right side or the left side of the driving path.

Here, the step of transmitting the information required for confirmation for confirming whether it can be prepared may further include the step of extracting the movement time information (a) generated based on the movement path and the movement means of the user instrument and transmitting it to the pharmacy terminal.

The identifying request information for confirming whether it can be prepared may include time information (b) capable of completing preparation that can complete preparation using prescribing information.

A pharmacy-matching system according to another aspect of the present invention comprises: a data transmitting/receiving unit for receiving the pharmacy search request information transmitted from the user instrument; a pharmacy information management department for extracting at least one pharmacy information included in the pharmacy search request information; a user management department that transmits at least one pharmacy information to the user instrument, requests prescribing information from the user instrument, and receives the information; and transmits the prescribing drug information included in the prescribing information to the pharmacy terminal included in the at least one pharmacy information to transmit information required for confirmation to confirm whether dispensing is possible and receive information on acceptance of whether dispensing is possible, and transmits at least one pharmacy information permitting the preparation to the user instrument by confirmation of the requested information.

Here, the pharmacy search request information includes current position information, destination information, and information on a movement means transmitted from the user instrument, and the pharmacy information includes a movement path of the user instrument generated using the current position information and destination information.

### Effects

The pharmacy-matching method according to an embodiment of the present invention allows a user to receive a prescription drug at a desired time at a pharmacy at a desired position. Since the drug can be prepared in advance, the convenience of both the user and the pharmacy can be increased.

In addition, since the pharmacist matching method according to an embodiment of the present invention can rationally adjust the cost of a user and the time for preparing a pharmacologic agent in a pharyngeal, it is possible to supply only the chemicals necessary for the preparation and use it in the preparation so as to not only increase the space utilization of the pharmacy but also decrease the inventory burden, thereby increasing the operating efficiency of the medicine.

The effects of the present invention are not limited to those mentioned above, and other effects not mentioned may be clearly understood by those skilled in the art from the following description.

### BRIEF DESCRIPTION OF THE DRAWINGS

FIG. 1 is a system block diagram of a service server for providing a pharmacy-matching method according to an embodiment of the present invention.

FIG. 2 is a flow diagram of a pharmacy-matching method according to an embodiment of the present invention.

FIG. 3 is a schematic diagram of a pharmacy-matching system according to an embodiment of the present invention.

FIG. 4 is a first illustration showing a display portion of a user instrument when using a pharmacy-matching system according to an embodiment of the present invention.

FIG. 5 is a second illustration showing a display portion of a user instrument when using a pharmacy-matching system according to an embodiment of the present invention.

### BEST MODE

Objects and effects of the present invention and technical configurations for achieving them will become apparent with reference to embodiments described in detail below in conjunction with the accompanying drawings. In the following description of the present invention, known functions or configurations will not be described in detail when it is determined that the gist of the present invention may be unnecessarily obscured thereby. The following terms are defined in consideration of the functions in the present invention and may vary depending on the intentions or customs of a user or operator.

However, the present invention is not limited to the embodiments disclosed below and may be implemented in various other ways. The embodiments are provided so that the disclosure of the present invention will be thorough and complete and will fully convey the scope of the invention to those of ordinary skill in the art. The scope of the present invention is only defined by the claims. Therefore, definitions should be based on the overall content of this specification.

Throughout the specification, when a part is referred to as “including” or “having” a certain component, this does not preclude other components and units that other components may further be included unless stated to the contrary. Also, terms, such as “unit,” “part,” and “module,” refer to units for processing at least one function or operation, and such units may be implemented with hardware, software, or a combination of hardware and software.

Meanwhile, in exemplary embodiments of the present invention, each of components, functional blocks, or units may include one or more sub-components, and electrical, electronic, and mechanical functions performed by the components may be implemented as various known devices or mechanical components including an electronic circuit, an integrated circuit, an application specific integrated circuit (ASIC) and the like. The components may be separately implemented, or two or more of the components may be integrated into one.

In exemplary embodiments of the present invention, a user device unit any calculation unit for collecting, reading, handling, processing, storing, and displaying data such as a desktop computer, a laptop computer, a smart phone, a personal digital assistant (PDA), and a cellular phone. In particular, the user device in exemplary embodiments of the present invention is a device having a function of executing software written in interpretable code and displaying and transferring the software being executed to a user. Also, as necessary, the user device may store the software therein or read the software together with data from the outside.

Also, the user device in exemplary embodiments of the present invention may include not only the above data processing function but also functions of input, output, storage, and the like. To this end, the user device may include not only various elements, such as a central processing unit (CPU), a mainboard, a graphics card, a hard disk drive, a sound card, a speaker, a keyboard, a mouse, a monitor, a universal serial bus (USB) terminal, and a communication modem, that general computing devices have but also a CPU, a mainboard, a graphics chip, a memory chip, a sound engine, a speaker, a touchpad, an external connection terminal, such as a USB terminal, a communication antenna, a communication modem for performing third generation (3G), Long Term Evolution (LTE), LTE-advanced (LTE-A), Wi-Fi, Bluetooth, etc. communication, and the like that wireless smart phone terminals have. Such elements may be used alone or in combination of two or more or parts of the elements may be combined to implement one or more functions. Devices, which are illustrated as one or more blocks in the drawings or detailed description according to exemplary embodiments of the present invention, or parts of the devices may represent one or more functions which are provided by various elements included in the user device alone or in combination of two or more or combined parts of the elements.

Meanwhile, in exemplary embodiments of the present invention, the user device, a multi-sports simulation apparatus, etc. may have a communication function and have various networking units, such as wired Internet, wireless Internet, infrared communication, Bluetooth, wideband code division multiple access (WCDMA), wireless broadband (WiBro), Wi-Fi, LTE, LTE-A, 3G, fourth generation (4G), fifth generation (5G), and a wired or wireless telephone network, to perform the communication function.

Hereinafter, a pharmacy-matching method according to the exemplary embodiment of the present invention will be described in detail with reference to the accompanying drawings.

A pharmacy-matching method according to an embodiment of the present invention comprises: a data transmitting/receiving unit of a service server receives a pharmacy search request information transmitted from a user instrument; a pharmacy information management department of the service server extracts at least one pharmacy information included in the pharmacy search request information; a user management department of a service server transmits at least the pharmacy information to a user instrument, the user management department requests the prescribing information to the user instrument and receives the information, the matching pharmacy management department of the service server transmits the prescribing drug information included in the prescription information to the pharmacy terminal included in at least one pharmacy information, thereby transmits information required for confirmation to confirm whether it can be prepared, and receives acceptance information about whether it can be prepared, and the matching pharmacy management department confirms the requested information to transmit at least one pharmacy information permitting the preparation to the user instrument.

The pharmacy search request information includes current position information, destination information, and information on movement means transmitted from the user instrument, the pharmacy information management department extracts pharmacy information within a predetermined radial range of a movement path of a user instrument generated using current position information and destination information, the movement means included in the information about the movement means is an automobile, public traffic, a bicycle, or a walking, and when the movement means is a public traffic, it is arranged to extract pharmacy information using point of departure, destination or transfer information of user instrument, and when the movement means is an automobile, it extracts pharmacy information using the driving path of the automobile, but selects and extracts pharmacy information located on the right side or the left side of the driving path.

The prescribing information is prescription image information or prescription text information, the step of requesting and receiving said prescribing information further comprises: if the prescribing information is the prescription image information, the service server transmits the prescription image information to the optical character recognition system server, requests to extract prescribing drug information to extract the prescribing drug information included in the prescription image information from the optical character recognition system, and receives the extracted prescribing drug information.

Here, the step of transmitting the identifying request information for confirming whether it can be prepared may further comprises the step of extracting the movement time information (a) generated based on the movement path and the movement means of the user instrument, and transmitting it to the pharmacy terminal.

Here, the information required for confirming whether or not the preparation may include time information (b) capable of completing preparation that can complete preparation using prescribing information,

A pharmacy-matching system according to another embodiment of the present invention comprises: a data transmitting/receiving unit for receiving the pharmacy search request information transmitted from the user instrument, a pharmacy information management department for extracting at least one pharmacy information included in the pharmacy search request information, the user management department for transmitting at least one pharmacy information to the user instrument and receiving the information by the user management department, and matching pharmacy management department for transmitting prescribing drug information included in the prescribing information to pharmacy terminal included in the at least one pharmacy information to transmit an information required for confirmation to confirm whether it can be prepared and to receive an acceptance information about whether it can be prepared, and transmits at least one pharmacy information permitting the preparation to the user instrument by confirmation of the requested information;

The pharmacy search request information includes current position information, destination information, and information on movement means transmitted from the user instrument, the pharmacy information management department extracts pharmacy information within a predetermined radial range of a movement path of a user instrument generated using current position information and destination information, the movement means included in the information about the movement means is an automobile, public traffic, a bicycle, or a walking, and when the movement means is a public traffic, it is arranged to extract pharmacy information using point of departure, destination or transfer information of user instrument, and when the movement means is an automobile, it extracts pharmacy information using the driving path of the automobile, but selects and extracts pharmacy information located on the right side or the left side of the driving path.

A pharmacy-matching method according to another embodiment of the present invention comprises: a data transmitting/receiving unit of the service server receives pharmacy search request information including current position information, destination information, and information on a movement means, transmitted from a user instrument; extracting, by the pharmacy information management department of the service server, pharmacy information within a predetermined radius of the movement path of the user instrument generated using at least one current position information and destination information included in the pharmacy search request information; transmitting, by a user management department of the service server, at least one pharmacy information to the user instrument; receiving, by the user management department, requesting prescribing information from a user instrument and receiving the information; a movement time information (a) generated on the basis of the movement path and the movement means of the user device at the periphery of the periphery included in the matching pharmacist management unit of the service server, transmitting and receiving acceptable information about whether or not the identifying request information is available for extracting and transmitting the prescribing drug information included in said prescribing information,

the information required for confirmation for confirming whether it can be prepared includes a time information (b) capable of completing preparation that can complete preparation using the prescribing information, the pharmacy terminal compares the movement time information (a) with the delivery time information of the distribution received from the third drug supply logistics system server that receives and delivers the specific drug, and when the movement time included in the movement time information (a) is longer than the preparation completion time, the information is transmitted to a service server to enable preparation of a prescription drug in accordance with the information required for confirmation, and the received matching pharmacy management department transmits additional preparable information to the user instrument via data transmitting/receiving unit.

The prescribing information is prescription image information or prescription text information, the step of requesting and receiving said prescribing information further comprises: if the prescribing information is the prescription image information, the service server transmits the prescription image information to the optical character recognition system server, requests to extract prescribing drug information to extract the prescribing drug information included in the prescription image information from the optical character recognition system, and receives the extracted prescribing drug information.

Here, the movement means included in the information on the movement means may be a automobile, public traffic, bicycle, or walking.

wherein the step of extracting the pharmacy information within a predetermined radius is provided to extract the pharmacy information by using the point of departure, destination, or transfer information of the user instrument when the movement means is public traffic.

wherein the step of extracting the pharmacy information within a predetermined radius range comprises extracting the pharmacy information by using the driving path of the automobile, when the movement means is a automobile, but selecting pharmacy information located on the right side or the left side of the driving path.

A pharmacy-matching service system according to another embodiment of the present invention includes: a data transmitting/receiving unit for receiving pharmacy search request information including current position information, destination information, and movement means transmitted from the user instrument; a pharmacy information management department configured to extract pharmacy information within a predetermined radius of a movement path of a user instrument generated using at least one current position information and destination information included in the pharmacy search request information; a user management department that transmits at least one pharmacy information to the user instrument, requests prescribing information from the user instrument, and receives the information; and a matching pharmacy management department of the service server for transmitting, to the pharmacy terminal included in at least one pharmacy information, movement time information (a) generated based on a movement path and a movement means of the user instrument by extracting the prescribing drug information included in said prescribing information, and transmits at least one pharmacy information permitting the preparation to the user instrument by confirmation of the requested information;

The information required for confirmation for confirming whether it can be prepared includes a time information (b) capable of completing preparation that can complete preparation using the prescribing information, the pharmacy terminal compares the movement time information (a) with the delivery time information of the distribution received from the third drug supply logistics system server that receives and delivers the specific drug, and when the movement time included in the movement time information (a) is longer than the preparation completion time, the information is transmitted to a pharmacy matching service system to enable preparation of a prescription drug in accordance with the information required for confirmation, and the received matching pharmacy management department transmits additional preparable information to the user instrument via data transmitting/receiving unit.

According to another embodiment of the present invention, there is provided a computer-readable recording medium in which a program for performing a pharmacy-matching method is stored.

### Embodiments

Objects and advantages of the present invention, and technical configurations for achieving them, will become apparent with reference to the embodiments described in detail below in conjunction with the accompanying drawings. In the description of the present invention, when it is determined that a specific description of a known function or configuration may unnecessarily obscure the gist of the present invention, the detailed description thereof will be omitted. And the following terms are defined terms in consideration of the donation in the present invention, which may vary according to the user, the intent or practice of the operator, etc.

However, the present invention is not limited to the embodiments disclosed below, but may be embodied in various different forms. The examples are merely provided to complete the disclosure of the present invention and to fully illuminate the scope of the invention to those skilled in the art, and the invention is only defined by the scope of the claims. The definition should therefore be made on the basis of the disclosure throughout this specification.

Throughout the specification, when a portion is said to “comprise” or “include” a component, this means that the component may further comprise other components but not other components unless specifically stated to the contrary.

The following provides a detailed description of a pharmacy-matching method according to an embodiment of the present invention with reference to the accompanying drawings.

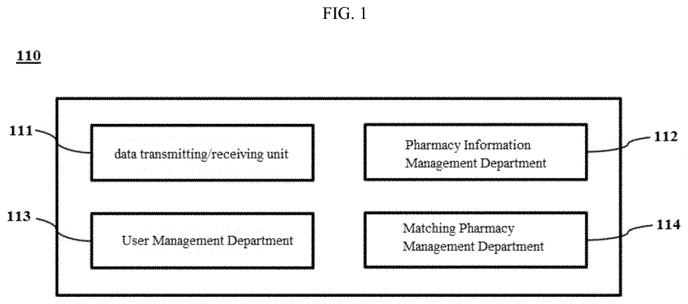

FIG. 1 is a system block diagram of a service server for providing a pharmacy-matching method according to an embodiment of the present invention.

The pharmacy-matching system according to an embodiment of the present invention may be configured to comprise a data transmitting/receiving unit, a pharmacy information management department, a user management department, and a matching pharmacy management department.

The data transmitting/receiving unit may be provided to enable wired/wireless data communication between the service server and the user instrument, and between the service server and the pharmacy terminal. The data transmitting/receiving unit may be transmitted directly or indirectly to a pharmacy information management department, user management department, matching pharmacy management department, as needed, and used to perform a pharmacy-matching method.

The pharmacy information management department may be provided to extract one or more pharmacy information included in the pharmacy search request information transmitted from the user instrument using pharmacy information transmitted by a storage device equipped with a service server in which the pharyngeal information is stored or a data communication method with an external server.

The pharmacy information can include, but is not limited to, position, name, address, etc. of the pharmacological country, and information provided from an open application program interface (open API) can be included without limitation.

A user management department may be provided to store or process information about the user of the user instrument. A user management department may be provided to request and receive prescribing information that includes information about a prescription or pharmaceutical product that desires the formulation with the user instrument.

The matching pharmacy management department may be provided to store, process, or transmit data such that, when prescribing information is exchanged between a user instrument and a pharmacy terminal, certain information may be filtered or matched between the pharmacy and user that suit conditions.

For example, a matching pharmacy management department may be provided to transmit a prescribing drug information included in the prescription information to a terminal of the drug, and store, process, and transmit the data to be used to receive and identify the request information to identify whether or not the prescribing drug is transmitted from the terminal of a drug. In addition, a user can be provided to receive the information required for confirmation for preparation of a prescribed drug from the user and transmit the information to a pharmacy terminal side, and to be able to identify the user by transmitting a physician of acceptability associated with preparation of the drug in the pharmacy to the user instrument by transmitting the declaration of intention thereto.

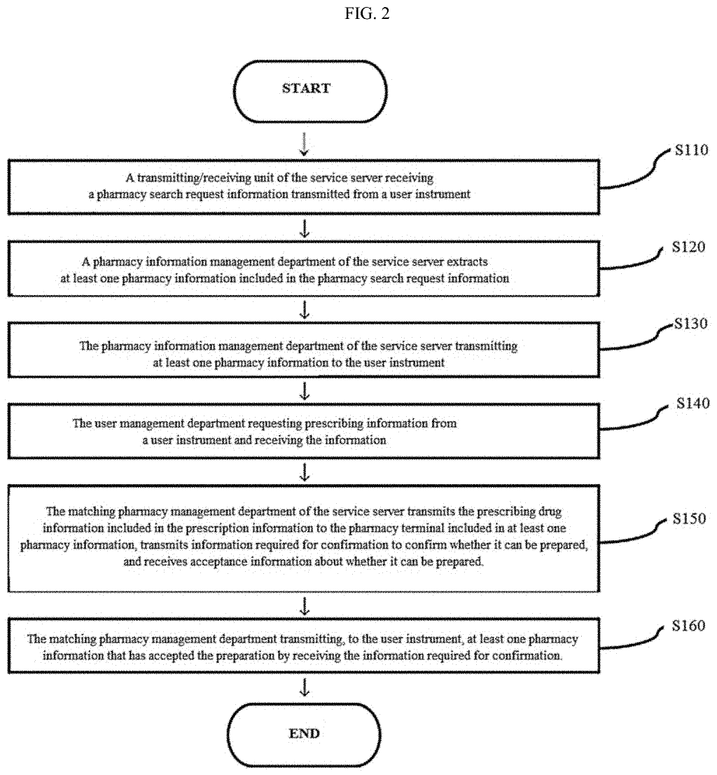

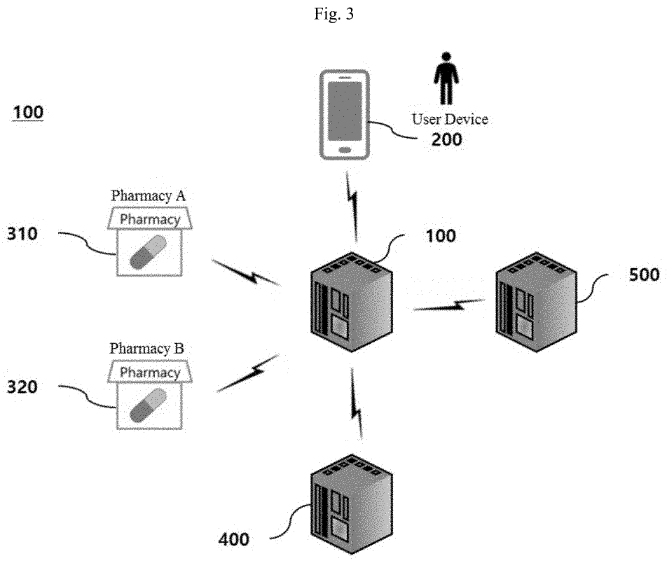

FIG. 2 is a flow diagram of a pharmacy-matching method according to an embodiment of the present invention, and FIG. 3 is a schematic diagram of a pharmacy-matching system according to embodiments of the invention.

Referring now to FIGS. 2 and 3, a pharmacy-matching method according to an embodiment of the present invention will be described in detail.

First, a pharmacy-matching method according to an embodiment of the present invention includes the step of receiving pharmacy search request information transmitted from a user instrument by data transmission and reception of service server (S110).

By this step, the user can transmit the pharmacy search request information to the service server through a user instrument that can transmit it to the user server. At this time, the user instrument may be a smart phone, a tablet computer, a personal digital assistant (PDA), or the like, but it may be used without limitation as long as it is a device that includes a computing function capable of wireless data communication.

Taking the case where a smartphone is used as a user instrument as an example, the user selects a desired position in a geographic information-providing image (indicator) expressed on a screen of a smartphone equipped with a touch screen, and the information is transmitted to a service server through a data receiver of the smartphone. A menu may be chosen to provide a user interface to determine whether to request a pharmacy search for that position, or to command the geographic information of a particular area to perform a search in a displayed state, or may be moved to a particular region and automatically provided with a phagocytic search request to transmit the pharmacy search request information to a service server after a pre-set predetermined time has elapsed.

Users may prefer a pharmacy near their home. In addition, the user of the prescribing drug may be a user, but is not limited thereto, and a person in a family relationship with the user. In this case there is a case where a prescription is desired in the pharmacy near the home in which the family resides.

In consideration of this situation, a user can store his personal information or the like in a user management department of a service server in advance and automatically set the pharmacy near the home of the user to be included in a pharmacy search request information or set it so as to include the pharyngeal request information of a family related to a prescribed drug.

Next, a pharmacy-matching method according to an embodiment of the present invention includes the step of extracting at least one pharmacy information included in the pharmacy search request information of a service server (S120).

The user instrument may be equipped with a Global Fositioning System (GPS) device, or equipped to generate its positional information using a GPS signal, wireless wireless communication, various networks, etc. The user instrument can use position information to define the user's current position information and transmit it to a service server.

The user instrument may include a navigation function to request mobile information to a particular destination using a map displayed on the display device. Through the navigation function, the user can also move to a specific destination, or provide a movement time and a movement path according to the movement means, such as when using public traffic or when using a passenger automobile for self-use.

The current position of the user instrument, the destination designated by the user, the movement means and the like are transmitted to the service server, and can be included in the pharmacy search request information as information about the current position information, destination information and movement means, respectively.

Since such pharmacy search request information includes information about current position information, destination information, and movement means transmitted from a user instrument, a pharmacy information management department can extract pharmacy information relating to such information.

For example, the step of the pharmacy information management department extracts pharmacy information may further comprise extracting pharmacy information within a predetermined radial range of the movement path of the user instrument generated using current position information and destination information. If the user moves from the current position A to the destination B, it is possible to choose the movement means and transmit information thereto to the service server.

The user's movement path may be represented by lines or the like in a map on the user instrument, wherein the pharmacy information management department of the service server extracts only a pharmacy information that is at a constant distance from the line represented by the movement path and is transmitted to the user instrument, and the user instrument may display only such pharmacy information so that the user can easily find a pharmacology on the path moving. In this case, the pharmacy information management department may be provided to pre-set the distance from the line represented by the travel path according to the movement means selected by the user. In this case, the movement means included in the information on the movement means may be a automobile, public traffic, bicycle, or walking.

For example, when moving by automobile, it can be preset as a pharmacy within a range such as a radius of 100 to 500 m from the moving path. If a user moves with a walk, it may be provided to provide the user with the setting of the pharmacopeia at a position where the distance from the line represented by the travel path does not deviate from a distance of approximately 100 to 200 m. That is, it can be set in advance so that the time required for the movement of the user can be appropriately adjusted according to the type of the movement means. However, it is not limited thereto and may be provided to arbitrarily select a distance from a line represented by a moving path according to the user's convenience.

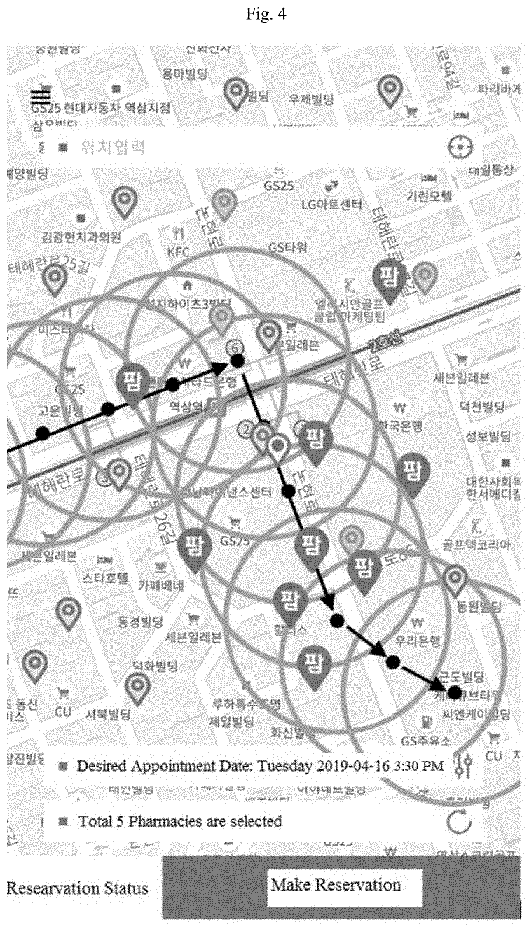

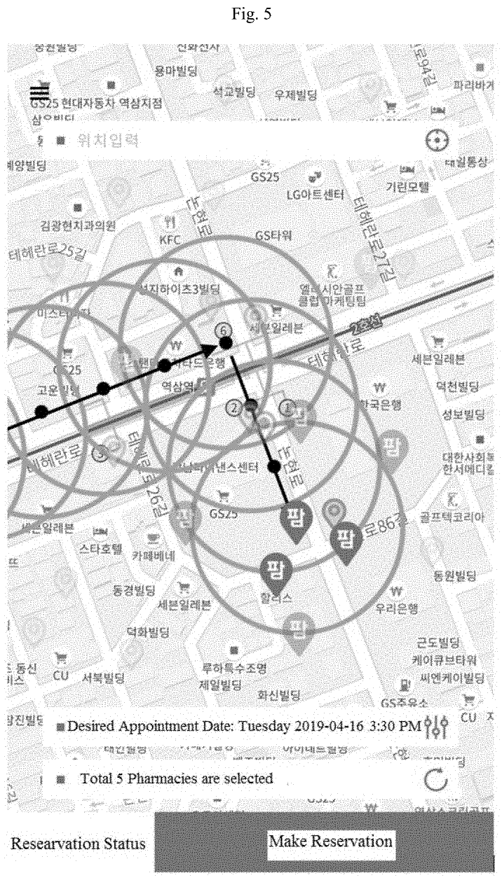

FIG. 4 is a first illustration diagram shown in a display section of a user instrument when using a pharmacy-matching system according to an embodiment of the present invention, and FIG. 5 is a second illustration showing a display portion of a user instrument when using a pharmacy-matching system according to an embodiment of the present invention.

FIG. 4 is a schematic representation of the movement path of a user and of the pharmacy information at a constant distance in a pharmacy-matching method according to an embodiment of the present invention. Referring to FIG. 4, there is shown a movement path of a user represented by a user's line and a circle of a predetermined radius so as to be displayed on a display part on a user instrument, so that a pharmacy with high user accessibility can be easily identified. The matching pharmacy management department may be provided to receive radial setting information to set a predetermined radius from a user instrument, filter the pharmacy information and transmit it to the user instrument.

On the other hand, when the matching pharmacy management department filters out the pharmacy information to provide the user with the pharmacy information, the user convenience can be increased by increasing the visibility of the user by displaying a predetermined radius along the moving path as a circle, displaying a portion other than that corresponding to the area of the circle in a blurred manner, or displaying a pharmacy close to the movement path of a user with high resolution. Referring to FIG. 5, it can be seen that the movement path of the user is represented by a solid line, in the case of the pharmacies located at a predetermined radius from the solid line is represented in a dark color, and is represented gradually more blurred depending on the distance from the travel path out of the predetermined radius, so that the user can more clearly recognize a pharmacy on the path.

Furthermore, the step of extracting the pharmacy information within a predetermined radial range from the movement route of the user may be provided with additional convenience information depending on how to define the movement means. For example, when the movement means is public traffic, it may be provided to extract the pharmacy information using the point of departure, destination, or transfer information of the user instrument.

At this time, the transfer information or the like can be used by receiving information about the public traffic transfer and the like from a public traffic system server for providing the information of the public traffic, or it is possible to use the public traffic transfer information that is predefined in a data storage part provided on a service server.

Even if a user is a pharmacy on a moving path, it is possible to increase the user's convenience by excluding the pharmacy where it takes a lot of time to gain access from the start paper, the work station, the rectifier located at the destination, etc.

Alternatively, when the movement means is an automobile, it may be provided to extract the pharmacy information using the driving path of the automobile, by selecting and extracting the pharmacy information located on the right or left side of the driving path. In the case of Korea as an example, it is very difficult to access the pharmacy on the opposite side beyond the center line even if the movement path is within a predetermined range since the bus center automobile changing is performed. Therefore, when the case of proceeding to the right side of the roadway satisfies the case where the central vehicle is implemented, the user convenience can be increased by making it possible to display only the pharmacies located on the right side of the roadway. Furthermore, in the case of an automobile, it can be provided to extract only the pharmacy information contained with information about whether or not a parking lot is possible, so that the user can be briefly lowered from the automobile to prescribe the medicine.

Next, a pharmacy-matching method according to an embodiment of the present invention includes the step of transmitting at least one pharmacy information to a user instrument by a user management department of a service server (S130.

There may be one or more pharmacies that meet pharmacy search request information on a user's movement path, and a pharmacy information management department may extract only the pharmacy information within a certain range from the movement path set as in the previous example and transmit it to a user instrument, thereby allowing the user to more easily use the pharmacy upon movement.

The user can determine the pharmacy information transmitted by the user management department and select whether to prepare a prescribing drug via a certain pharmacy.

Next, a pharmacy-matching method according to an embodiment of the present invention includes a user management request and receiving the prescribing information to a user instrument (S140).

In general, in the case of a drug prescribed in a hospital at a specific position, it is easy to prepare a prescribed drug in a pharmacy that is closely adjacent to the hospital at the specific position in a geographic manner, but in the case of a drug prescribed in another area, the drug according to the prescription may not be provided. According to this step, a user management department may request prescribing information from the user instrument, wherein the prescribing information may be prescription image information or prescription text information. The prescription image information may be a bar code, a QR (Quick Response Code), or the like, but is not limited thereto and may be used as long as it is not capable of compressing and redirecting certain information. The text information includes direct input prescription information as well as prescription information through Optical character recognition (OCR) which converts the machine to readable characters.

In addition, the step of request and receiving the prescription information may further include, when the prescribing information is prescription image information, for example, when information such as a text image or a QR code image transmitted from a user's user instrument is transmitted to a service server, the service server transmits the prescription image information to an optical character recognition system server, and request and receive the extracted prescribing drug information from the optical character confirmation system server to extract the prescribing drug information included in the prescription image data by a method such as optical character recognition.

The prescribing information is preferably provided in the form of text or the like to the pharmacy terminal and the like, to provide convenience to directly identify and prescribe the pharmacy agent. However, the present invention may include, but is not limited to, a case in which a prescription image or the like containing prescribing information can be automatically updated to read the whether or not a drug is retained or inventory in a pharmacy terminal itself.

Even if the prescribing information is in the form of text, it may have to be used in a printed state on the paper, and the user may collect the prescription text and the like through a camera or the such equipped with a user instrument and transmit it to a service server.

The service server includes optical character recognition (OCR) or can be used as prescribing drug information by reading the prescription text through data transmission and transmission with an optical character confirmation system server equipped with an URI and converting it into a form of data that can be edited and processed on a computer.

The prescribing information may include personal information of the user, etc., in addition to including prescribing drug information. Depending on the legislation, it may constitute the requirement of illicit action that personal information included in the prescribing information is provided to use a third party without allowing the user. Thus, a user management department may be provided to extract only the prescribing drug information included in the prescription information and such prescribing drugs information may be transmitted to a matching pharmacy management department and transmitted to the pharmacy terminal.

Next, the matching pharmacy management department of the service server transmits the prescribing drug information included in the prescription information to the pharmacy terminal included in at least one pharmacy information, transmits information required for confirmation to confirm whether it can be prepared, and receives acceptance information about whether it can be prepared (S150).

At least one pharmacy located in the path of movement by the user or in the particular area selected can be represented in the display section of the user instrument in order to transmit the prescribing drug information to each pharmacy and confirm whether it is pre-formulated.

When it has been confirmed whether or not it can be prepared in advance, a matching pharmacy management department can transmit the confirmation data together to allow confirmation of a false part according to whether it is prepared by a user instrument.

Unlike in the past, the water jet system is operated at a high speed in the case of a dozen paper. If a user receives a prescription of a drug during the designated and moving destination, a constant time is consumed by the moving distance. Therefore, if a prescribing drug can be included during the time of the movement being consumed, it can be switched to a case where a specific drug is delivered by the drug supply logistics system server 500 and a preparation is possible even if the preparation is impossible when receiving the original information required for confirmation.

Therefore, it may be possible to confirm whether it can be prepared in consideration of the movement time of the user.

For example, the step of transmitting the identifying request information for confirming whether it can be prepared may further comprises the step of extracting the movement time information (a) generated based on the movement path and the movement means of the user instrument, and transmitting it to the pharmacy terminal.

If the movement time according to movement time information (a) takes a longer time than the time at which the drug can be delivered from the drug supply logistics system server (500), the preparation of the drug during the movement of the user can be changed to a possible state.

Accordingly, the information required for confirming whether or not the preparation may include time information (b) capable of completing preparation that can complete preparation using prescribing information,

When the movement time information (a) to the pharmacy exceeds a predetermined time range preset in advance than the time information (b) capable of completing the preparation, the pharmacy terminal can be transmitted to the service server as being capable of preparing the prescribing drug according to the information required for confirmation, and the matching pharmacy management department can provide additional preparable pharmacy information to the user instrument.

Furthermore, since the user convenience can be further increased if the time required to complete the actual preparation can be estimated rather than considering only the delivery time of logistics and the preparation time of the pharmacy can be prepared in advance, information about logistics time and the production time of a pharmacy agent can be used to generate time information (b) capable of completing the preparation.

Specifically, there are cases in which the user has to move after receiving medical treatment at the hospital. If a margin occurs after the end of a certain event, it must move along a new path, but it may be the case that it goes back to the vicinity of the prescription hospital for the medication. For example, if you receive a prescription for a drug, take your child to kindergarten, and want to find a prescription medicine when you have free time, the time interval from issuing a prescription to requesting the actual dispensing may occur relatively large.

Alternatively, users who have regular pharmacies may want to have their regular pharmacies dispense medications according to prescriptions, but may not have stock. In this case, the user may arbitrarily delay the preparation request time.

As described above, it may be impossible to prepare the drug at a specific site at a particular time. It is necessary to determine the time required for the preparation and to find it.

When the movement time information (a) to the pharmacy exceeds a predetermined time range preset in advance than the time information (b) capable of completing the preparation, such as in the case of event occurrence or when the use of the frequenter pharmacy is desired, the pharmacy terminal can be transmitted to the service server as being capable of preparing the prescribing drug according to the information required for confirmation, and the matching pharmacy management department can provide additional preparable pharmacy information to the user instrument. Next, the pharmacy-matching method according to an embodiment of the present invention then includes that the matching pharmacy management department confirms the requested information to transmit at least one pharmacy information permitting the preparation to the user instrument (S160).

Previously, there may be cases where it is difficult to substantially prepare and supply a drug, such as in the case where a lot of hands are in the atmosphere or in the absence of the drug, in order to prepare the actual drug in the pharmacy in which the information required for confirmation is confirmed. Therefore, it is possible to confirm the information required for confirmation and transmit the information to a user instrument by accepting the preparation only in the case of the pharmacy transmitted to the service server by the declaration of intention of acceptance, so that accurate information is transmitted to the user.

In this case, among the pharmacy that has identified and accepted the information required for confirmation, the information that has been the most quickly accepted of the pharmacy can be sent to the user instrument in preference. This may allow efficient schedule control as the user may take into account the time, distance, etc. of movement.

On the other hand, If a user specifies a specific pharmacy as formulating a drug, the matching pharmacy management department transmits a information required for confirmation deletion signal to the pharmacy terminal so that the information required for confirmation disappears from the remaining pharmacy terminals, thereby preventing the accumulation of unnecessary data. In addition, when a physician's prescription is issued, it is effective only for a certain period of time from the date of the prescription publication. When the expiration date of the prescription is exceeded, it may also be provided that the deletion signal of information required for confirmation is transmitted to the pharmacy terminal and the information required for confirmation is lost in the pharmacy terminal.

According to another embodiment of the present invention, there may be provided a pharmacy-matching system comprising: a data transmitting/receiving unit for receiving the pharmacy search request information transmitted from the user instrument, a pharmacy information management department for extracting at least one pharmacy information included in the pharmacy search request information, the user management department for transmitting at least one pharmacy information to the user instrument and receiving the information by the user management department, and matching pharmacy management department for transmitting prescribing drug information included in the prescribing information to pharmacy terminal included in the at least one pharmacy information to transmit an information required for confirmation to confirm whether it can be prepared and to receive an acceptance information about whether it can be prepared, and transmits at least one pharmacy information permitting the preparation to the user instrument by confirmation of the requested information;

In addition, according to another embodiment of the present invention, a computer-readable recording medium may be provided in which a program for performing the following is stored: a data transmitting/receiving unit of a service server receives a pharmacy search request information transmitted from a user instrument, a pharmacy information management department of the service server extracts at least one pharmacy information included in the pharmacy search request information, a user management department of a service server transmits at least the pharmacy information to a user instrument, the user management department requests the prescribing information to the user instrument and receives the information, the matching pharmacy management department of the service server transmits the prescribing drug information included in the prescription information to the pharmacy terminal included in at least one pharmacy information, thereby transmits information required for confirmation to confirm whether it can be prepared, and receives acceptance information about whether it can be prepared, and matching pharmacy management department confirms the required information and transmits at least one pharmacy information permitting the preparation to the user instrument.

While the specification and drawings have described preferred embodiment of the present invention, certain terms have been used, they are used only in the generic sense to facilitate explanation and understanding of the invention, and are not intended to limit its scope It will be apparent to those skilled in the art that other modifications based on the technical concept of the present invention can be carried out in addition to the embodiments disclosed herein.

### DESCRIPTION OF NUMERICAL REFERENCES IN THE FIGS

10: pharmacy-matching System
100: service server
111: data transmitting/receiving unit
112: Pharmacy Information Management Department
113: User Management Department
114: Matching Pharmacy Management Department
200: user instrument
310: A pharmacy
320: B pharmacy
400: Optical Character Recognition System Server
500: Drug Supply Logistics System Server

1. A pharmacy-matching method, comprising:
a transmitting/receiving unit of the service server receives a pharmacy search request information transmitted from a user instrument;
a pharmacy information management department of the service server extracts at least one pharmacy information included in the pharmacy search request information;
a user management department of the service server transmits at least one pharmacy information to the user instrument;
the user management department requests a prescribing information from a user instrument and receiving the information;
the matching pharmacy management department of the service server transmits the prescribing drug information included in the prescription information to the pharmacy terminal included in at least one pharmacy information, transmits information required for confirmation to confirm whether it can be prepared, and receives acceptance information about whether it can be prepared; and
the matching pharmacy management department confirms the requested information to transmit at least one pharmacy information permitting the preparation to the user instrument;
the pharmacy search request information includes user instrument's current position information, destination information, and information on movement means transmitted from the user instrument,
the step of extracting the pharmacy information further comprises extracting the pharmacy information within a predetermined radius of the movement path of the user instrument generated using the current position information and the destination information,
the movement means included in the information on the movement means is an automobile, public traffic, bicycle or walking,
when the movement means is public traffic, it is provided to extract pharmacy information by using point of departure, destination or transfer information of the user instrument, and
when the movement means is an automobile, the pharmacy information is extracted by using the driving path of the automobile, but selecting pharmacy information located on the right side or the left side of the driving path.

2. The method of claim 1, wherein the prescribing information is prescription image information or prescription text information,
the step of requesting and receiving said prescribing information further comprises: if the prescribing information is the prescription image information, the service server transmits the prescription image information to the optical character recognition system server, requests to extract prescribing drug information to extract the prescribing drug information included in the prescription image information from the optical character recognition system, and receives the extracted prescribing drug information.

3. The method of claim 1, wherein the step of transmitting the identifying request information for confirming whether it can be prepared further comprises the step of extracting the movement time information (a) generated based on the movement path and the movement means of the user instrument, and transmitting it to the pharmacy terminal.

4. The method of claim 3, wherein identifying request information for confirming whether it can be prepared includes time information (b) capable of completing preparation that can complete preparation using prescribing information.

5. A pharmacy-matching system, comprising:
a data transmitting/receiving unit for receiving the pharmacy search request information transmitted from a user instrument;
a pharmacy information management department for extracting at least one pharmacy information included in the pharmacy search request information;
a user management department that transmits the at least one pharmacy information to a user instrument, requests prescribing information from the user instrument, and receives the information; and
a matching pharmacy management department that transmits a prescribing drug information included in a prescribing information to a pharmacy terminal included in the at least one pharmacy information, transmits the identifying request information for confirming whether it can be prepared, and receives acceptance information about whether it can be prepared, and transmits at least one or more pharmacy information to a user instrument to accept a preparation,
the pharmacy search request information includes current position information, destination information, and information on movement means transmitted from the user instrument,
the pharmacy information management department extracts pharmacy information within a predetermined radial range of a movement path of a user instrument generated using current position information and destination information, the movement means included in the information about the movement means is an automobile, public traffic, a bicycle, or a walking, and when the movement means is a public traffic, it is arranged to extract pharmacy information using point of departure, destination or transfer information of user instrument, and when the movement means is an automobile, it extracts pharmacy information using the driving path of the automobile, but selects and extracts pharmacy information located on the right side or the left side of the driving path.

6. A pharmacy-matching method, comprising:
a data transmitting/receiving unit receives pharmacy search request information including current position information, destination information, and movement means transmitted from the user instrument;
a pharmacy information management department extracts pharmacy information within a predetermined radius of a movement path of a user instrument generated using at least one current position information and destination information included in the pharmacy search request information;
a user management department of the service server transmits at least one pharmacy information to the user instrument;
the user management department requests a prescribing information from a user instrument and receiving the information;
the matching pharmacy management department of the service server transmits, to the pharmacy terminal included in at least one pharmacy information, movement time information (a) generated based on a movement path and a movement means of the user instrument by extracting the prescribing drug information included in said prescribing information, transmits an identifying request information for confirming whether it can be prepared, and receives an acceptance information about whether it can be prepared; and
the matching pharmacy management department confirms the requested information to transmit at least one pharmacy information permitting the preparation to the user instrument;
the identifying request information for confirming whether it can be prepared includes time information (b) capable of completing preparation that can complete preparation using prescribing information,
the pharmacy terminal compares the movement time information (a) with the delivery time information of the distribution received from the third drug supply logistics system server that receives and delivers the specific drug, and when the movement time included in the movement time information (a) is longer than the preparation completion time, the information is transmitted to a service server to enable preparation of a prescription drug in accordance with the information required for confirmation, and the matching pharmacy management department of service server received transmits additional preparable information to the user instrument via data transmitting/receiving unit.

7. The method of claim 6, the prescribing information is prescription image information or prescription text information,
the step of requesting and receiving said prescribing information further comprises: if the prescribing information is the prescription image information, the service server transmits the prescription image information to the optical character recognition system server, requests to extract prescribing drug information to extract the prescribing drug information included in the prescription image information from the optical character recognition system, and receives the extracted prescribing drug information.

8. The method of claim 6, wherein the movement means included in the information on the movement means is an automobile, public traffic, bicycle, or walking.

9. The method of claim 8, wherein the step of extracting the pharmacy information within a predetermined radius is provided to extract the pharmacy information by using the point of departure, destination, or transfer information of the user instrument when the movement means is public traffic.

10. The method of claim 8, wherein the step of extracting the pharmacy information within a predetermined radius range comprises extracting the pharmacy information by using the driving path of the automobile, when the movement means is a automobile, but selecting pharmacy information located on the right side or the left side of the driving path.

11. A pharmacy-matching service system, comprising:
a data transmitting/receiving unit for receiving pharmacy search request information including current position information, destination information, and movement means transmitted from the user instrument;
a pharmacy information management department extracting pharmacy information within a predetermined radius of a movement path of a user instrument generated using at least one current position information and destination information included in the pharmacy search request information;
a user management department that transmits the at least one pharmacy information to a user instrument, requests prescribing information from the user instrument, and receives the information; and
a matching pharmacy management department transmits, to the pharmacy terminal included in at least one pharmacy information, the movement time information (a) generated based on a movement path and a movement means of the user instrument, and the prescribing drug information included in said prescribing information, thereby transmits an identifying request information for confirming whether it can be prepared, receives an acceptance information about whether it can be prepared, and transmits at least one or more pharmacy information to a user instrument to accept a preparation; and
the identifying request information for confirming whether it can be prepared includes time information (b) capable of completing preparation that can complete preparation using prescribing information,
the pharmacy terminal compares the movement time information (a) with the delivery time information of the distribution received from the third drug supply logistics system server that receives and delivers the specific drug, and when the movement time included in the movement time information (a) is longer than the preparation completion time, the information is transmitted to a pharmacy matching service system to enable preparation of a prescription drug in accordance with the information required for confirmation, and the received matching pharmacy management department transmits additional preparable information to the user instrument via data transmitting/receiving unit.

12. A computer-readable recording medium storing a program for performing the pharmacy-matching method according to claim 1.

13. A computer-readable recording medium storing a program for performing the pharmacy-matching method according to claim 6.
